(19) United States
(12) Patent Application Publication
(10) Pub. No.: US 2004/0087010 A1
(43) Pub. Date: May 6, 2004

(54) Micro ELISA reader

(76) Inventor: Chung-Hua Tsai, Hsinchu (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/286,832

(22) Filed: Nov. 4, 2002

(51) Int. Cl. C12M 1/34

(52) U.S. Cl. 435/287.2; 435/288.4; 435/288.7; 435/808

(57) ABSTRACT

A micro ELISA reader includes a housing, a tray longitudinally movably provided in the housing, a carrier transversally movably mounted on the tray, a light source board provided on the carrier, and a scanning assembly longitudinally movably mounted in the housing and above the light source board. The ELISA reader is integrally installed in a personal computer. During a testing, a microtiter plate is stably positioned in the carrier and between the light source board and the scanning assembly, and scanned by the scanning assembly.

### BACKGROUND OF THE INVENTION

1. Field of the Invention

The present invention is related to an ELISA reader, and more particularly a micro ELISA reader which can be integrally installed in a computer.

2. Description of Related Art

ELISA readers are devices for ELISA (Enzyme-Linked Immunosorbent Assay), which is a test method to measure an absorbency of a solution. The conventional ELISA readers are not only applicable for the special tests but also widely applied in many fields including color reactions of enzymes, dynamic experiments of enzymes, minimum inhibiting concentration of antibiotics, agglutination tests, complement fixing tests, minimum blood type assay, toxic reactions of monolayer cells, etc.

Each of the conventional ELISA readers has a microtiter plate made in accordance with a standard specification. A standard 96-well plate is the widest used microtiter plate, and most conventional ELISA readers have sizes and specifications compatible with the 96-well plate.

In testing and assaying, the ELISA reader is generally connected with a computer, and data read by the ELISA reader is transmitted into the computer for storing and analyzing. The ELISA reader and the computer will occupy a large space, and it is also inconvenient for a user to connect and operate the ELISA reader and the computer.

However, all of the conventional ELISA readers are large in size and cannot be directly installed in the computers.

Therefore, the invention provides a micro ELISA reader to mitigate and/or obviate the aforementioned problems.

### SUMMARY OF THE INVENTION

The main objective of the invention is to provide a micro ELISA reader which can be integrally installed in a personal computer.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a micro ELISA reader in accordance with the invention;

FIG. 2 is a perspective view of the micro ELISA reader installed in a personal computer;

FIG. 3 is a perspective of the micro ELISA reader in FIG. 1;

FIG. 4 is a side sectional view of the micro ELISA reader to scan a micro-titer plate;

FIG. 5 is a top view schematically showing a transversal movement of the micro ELISA reader; and

FIG. 6 is a top view schematically showing a longitudinal movement of the micro ELISA reader.

### DETAILED DESCRIPTION OF THE PREFERRED EMBODIMENT

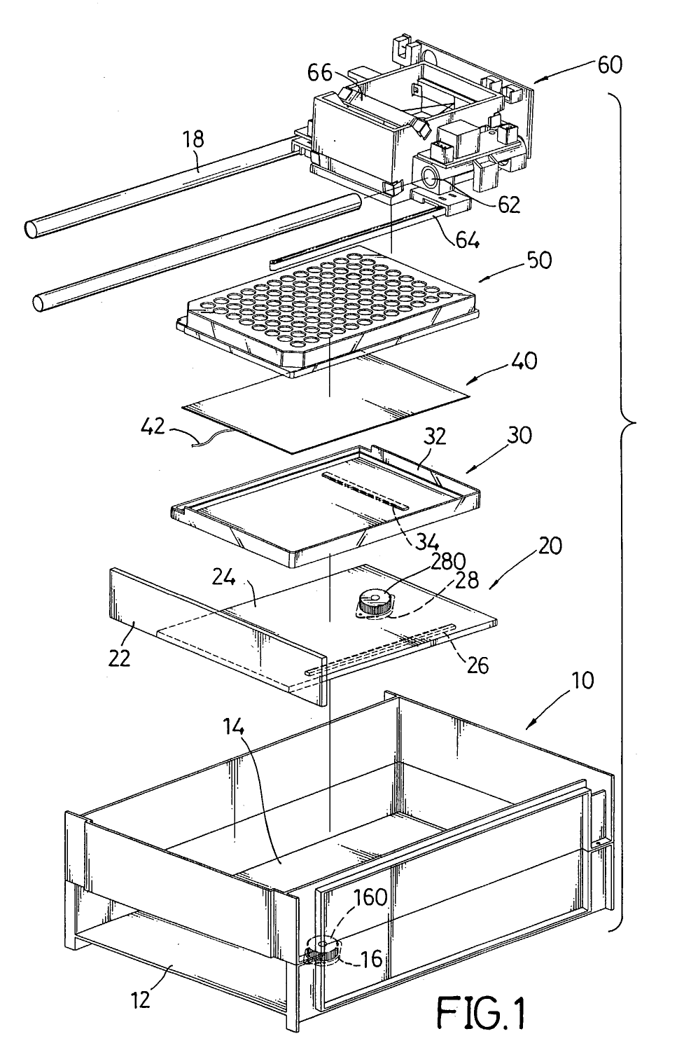

Referring to FIG. 1, a micro ELISA reader in accordance with the invention has a housing (10) being composed of an upper portion and a lower portion. An opening (12) is defined at a front side of the lower portion of the housing (10). A first gear (160) is provided on the bottom plate (14) and can be driven by a first motor (16) provided under the bottom plate (14). Two rails (18) are longitudinally provided in the upper portion of the housing (10).

A tray (20) is provided in the lower portion of the housing (10) and is longitudinally movable through the opening (12). The tray (20) has a bottom board (24) parallel to the bottom plate (14) of the housing (10), and a panel (22) perpendicular to the bottom board (24) formed at a front side thereof to close the opening (12) when the tray (20) is received in the housing (10). A second motor (28) is provided under the bottom board (24), and a second gear (280) is provided on the bottom board (24) and driven by the second motor (28). A first rack (26) is longitudinally provided under the bottom board (24) and engaged with the first gear (160) on the bottom plate (14). Thus, when the first gear (160) is driven by the first motor (16) to move the rack (26), the tray (20) can be longitudinally extended out from the opening (12) or retracted in through the opening (12).

A carrier (30) is mounted on the tray (20) and has a second rack (34) transversally provided at a bottom surface thereof and engaged with the second gear (280). Baffles (32) are formed at sides of the carrier (30). When the second motor (28) is actuated to drive the second gear (280), the carrier (30) can be transversally moved by the second rack (34) engaged with the second gear (280).

A light source board (40) is provided on the carrier (30) and inside the baffles (32). The light source board (40) has wires (42) electrically connected with an external power supply (not shown or numbered).

A 96-well plate (50) is placed on the light source board (40), and light emitted from the light source board (40) can pass through transparent wells of the plate (50).

A scanning assembly (60) is provided in the lower portion of the housing (10) and above the 96-well plate (50) with a vertical distance from the 96-well plate (50). Two holes (62) are longitudinally and respectively defined at two sides of the scanning assembly (60). The rails (18) are respectively inserted through the holes (62), and the scanning assembly (60) is longitudinally movable along the rails (18). A belt (64) is provided at one of the two sides of the scanning assembly (60) for driving the scanning assembly (60). A plurality of mirrors (66) is provided in the scanning assembly (60) to reflect light signals from the 96-well plate (50). The scanning assembly (60) further has a CCD (Charge-Coupled Device) light signal receiver (not shown or numbered) or the like to receive and process the light signals reflected by the mirrors (66). The structure of the scanning assembly is similar to the conventional products and will not be described in detail.

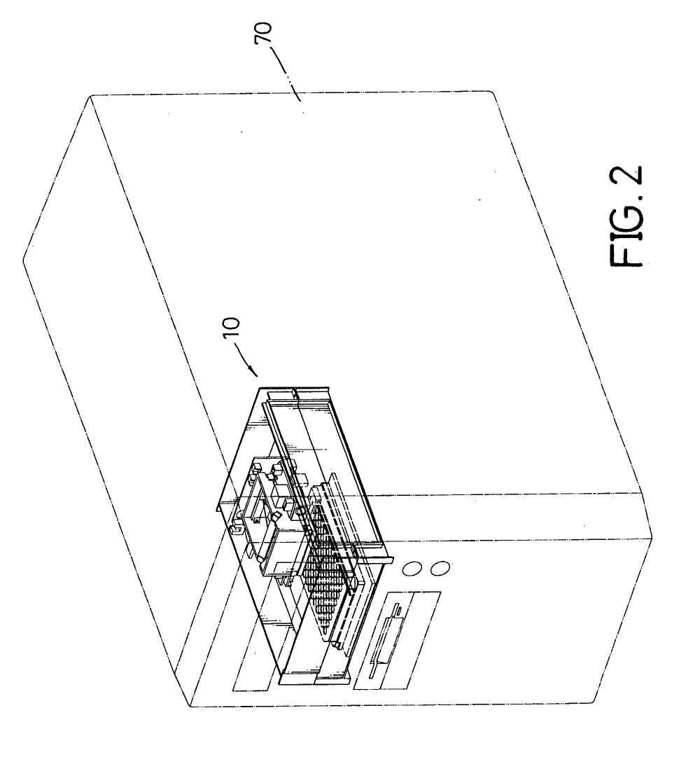

Referring to FIG. 2, the micro ELISA reader has external dimensions as a 5.25″ FDD (Floppy Disk Drive), so that the micro ELISA reader can be integrally installed in a 5.25″ standard slot of a personal computer (70), and data wires of the micro ELISA reader (not shown or numbered) can be directly connected to the computer.

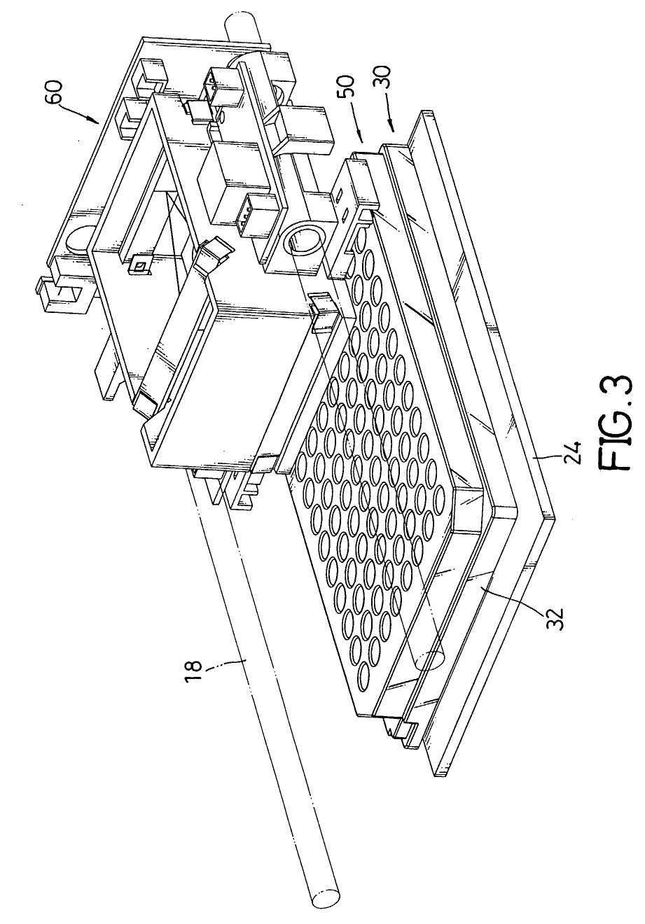

FIG. 3 illustrates the assembled micro ELISA reader. The size of the baffles (34) of the carrier (30) is designed to match the standard specification of the 96-well plate (50), so that the 96-well plate (50) can be stably positioned in the carrier (30).

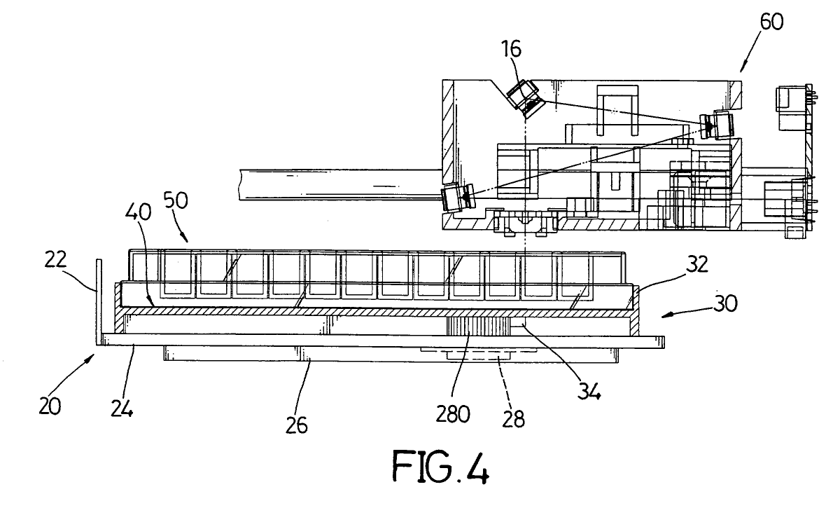

Referring to FIG. 4, during a testing, the 96-well plate (50) is filled with solutions and placed on the carrier (30), and the light source board (40) under the 96-well plate (50) emits light passing through the solutions in the 96-well plate (50). The light signals are reflected by the mirrors (66) to transmit into the light signal receiver and the computer for further processing and analyzing. According to these light signals, the absorbencies of the solutions can be measured.

Referring to FIG. 5, because a height of the micro ELISA reader is limited by the specification of the 5.25″ slot, the distance between the 96-well plate (50) and the scanning assembly (60) is so short that in one stroke of the scanning assembly (60) a maximum transversal scope scanned by each scanning member (not shown or numbered) of the scanning assembly (60) reaches only one half well.

By transversally moving the carrier (30), each well can be scanned completely through two strokes. In a forward stroke where the scanning assembly (60) is moved towards the front side of the housing (10), one half of every well is scanned. Thereafter, the second motor (28) drives the second gear (280) and the second rack (34) to transversally move the carrier (30). Then, in a backward stroke where the scanning assembly (60) is moved away from the front side of the housing (10), the other half of every well is scanned.

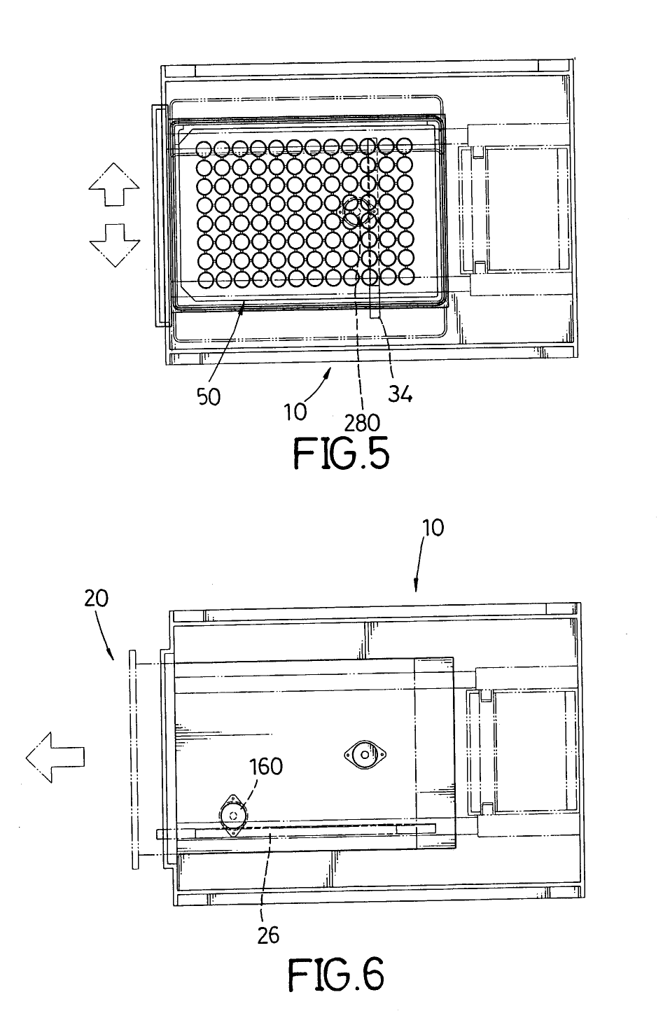

Referring to FIG. 6, the tray (20) can be ejected from the housing (10) by means of the first motor (16) driving the first gear (160) and the first rack (26) for replacing the 96-well plate (50).

According to the principle and teaching of the invention, any other type of microtiter plate which can adapt to the 5.25” slot of the computer is also usable in the micro ELISA reader if the size of the carrier (30) is modified to match the parameters of the used microtiter plate.

According to the present invention, the light source board is provided under the microtiter plate, so that the overall size of the ELISA reader is effectively minimized. Therefore, the ELISA reader can be directly installed in a 5.25″ slot of a personal computer to reduce an occupied space, and also enables easy and convenient operation by a user.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A micro ELISA reader comprising:
a housing (10) having a lower portion, an upper portion, an opening (12) defined at a front side of the lower portion, and two rails (18) longitudinally provided in the upper portion;
a tray (20) provided in the lower portion of the housing (10) and longitudinally movable out from the housing (10) through the opening (12);
a carrier (30) transversally movably mounted on the tray (20);
a light source board (40) provided on the carrier (30); and
a scanning assembly (60) mounted on the rails (18) and longitudinally movable along the rails (18), and above the light source board (40) with a vertical distance from the light source board (40),
whereby, the ELISA reader is integrally installed in a personal computer, and during a testing, a microtiter plate (50) is stably positioned in the carrier (30) and between the light source board (40) and the scanning assembly (60), and scanned by the scanning assembly (60).

2. The micro ELISA reader as claimed in claim 1, wherein the housing (10) has a first motor (16) provided under a bottom plate (14) thereof, and a first gear (160) provided on the bottom plate (14) and driven by the first motor (16); and
the tray (20) has a bottom board (24) parallel to the bottom plate (14), and a first rack (26) longitudinally formed under the bottom board (24) and engaged with the first gear (160).

3. The micro ELISA reader as claimed in claim 2, wherein the tray (20) has a second motor (28) provided under the bottom board (24), and a second gear (280) provided on the bottom board (24) and driven by the second motor (28); and
the carrier (30) has a second rack (34) provided at a bottom surface thereof and engaged with the second gear (280).

4. The micro ELISA reader as claimed in claim 1, wherein the tray (20) has a panel (22) perpendicular to the bottom board (24) formed at a front side thereof for closing the opening (12).

5. The micro ELISA reader as claimed in claim 1, wherein the carrier (30) has baffles (32) formed at sides thereof.
